(19) United States
(12) Patent Application Publication
(10) Pub. No.: US 2004/0038952 A1
(43) Pub. Date: Feb. 26, 2004

(54) Pharmaceutical compositions for treatment of digestive disorders and associated diseases

(76) Inventor: Janos Feher, Budapest (HU)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/450,359

(22) PCT Filed: Dec. 13, 2001
(86) PCT No.: PCT/HU01/00123

(30) Foreign Application Priority Data
Dec. 13, 2000 (HU) P0004919
Dec. 11, 2001 (HU) P0105291

(51) Int. Cl. A61K 31/56; A61K 31/7048; A61K 31/353

(52) U.S. Cl. 514/171; 514/456; 514/27

(57) ABSTRACT

The invention is related to the preparation of a pharmaceutical composition containing a bile acid component and a bioflavonoid component. The pharmaceutical composition according to the invention may be used for the treatment of digestive disoreders, systemic diseases associated with digestive disorders and diseases, furthermore for improving the digestion and absorption of the fat-soluble vitamins of foods, essential fatty acid and food components as well as food additives.

The invention relates to pharmaceutical composition for the treatment and prevent of digestive disorders, as well as diseases and disorders caused by digestive disorders and for the preparation thereof.

In the industrialized countries digestive disorders are mentioned among the most common diseases, which are the functional diseases of the digestive system (gastritis or nonulcer dyspepsia, colitis or irritable bowel disease, biliary-disorders or dyskensis etc). There are more and more evidences that the functional diseases of the gastrointestinal tract often precede or cause organic diseases (for example gastroduodenal ulcer, diverticulosis or diaveticulitis and cancer). Furthermore the digestive disorders are often associated with other diseases, such as mental disorders (anxiety, depression, panic disorder or so-called psychosomathic diseases), neurodegenerative diseases (Alzheimer disease, Parkinson disease, retinal diseases caused by age), cardiovascular diseases (such as atherosclerosis and similar diseases) or the degenerative diseases of the osseous and muscular system (such as osteoarthritis or athrosis), as well as immune and endocrine diseases. Neither of the cause of the digestive disorders nor the correlation between the digestive disorders and the neuro-immune-endocrine diseases are fully understood. Consequently, symptomatic methods are used for the treatment of the above diseases, mostly adequate diet in itself or associated with antidepressants-sedative treatment and eventually spasmolytic-pain releaving or antiinflammatory treatment. The disadvantage of these methods is that patients affected by digestive disorders may not tolerate most of these orally administered medicaments [Drossman, D. A (Senior Editor), The Functional Gastrointestinal Disorders, 2nd Edition, Degnon Ass., McLean, (2000)].

During the treatment of certain conditions or gallstones animal bile extracts and bile acids or the salts thereof are used for bile substitution. These, however, may cause unexpected side effects (such as diarrhea, altered transaminase values, even more liver damage). It is known that traditionally several mixtures of different bioflavonoid-containing plant extracts are used for improving digestive disorders. The above plant extracts show, however, only symptomatic, weak and transient effects when applied for the treatment of the above-mentioned diseases in the clinical practice.

The aim of our invention was to improve the effect of the known bile or bioflavonoid-containing pharmaceutical compositions.

The above aim was achieved by the pharmaceutical composition according to the invention.

The subject of the invention is a pharmaceutical composition containing a bile acid component and a bioflavonoid component as active ingredient associated with pharmaceutically active carriers and/or auxiliary agents.

As bile acid component the pharmaceutical composition of the invention may contain the following: bile acid or its conjugates (such as conjugates with taurine or glycine), bile acidic salts or semi-synthetic or synthetic analogues, preferably ursodeoxycholic acid, sodium-deoxycholate, tauro-deoxycholate, glyco-deoxycholate, sodium-ursodeoxycholate, glyco-ursodeoxycholate or tauro-ursodeoxycholate etc; furthermore full animal bile or the animal bile extract in a solvent or dry extract of animal bile or any fraction containg bile acid. The full animal bile or the dry bile extract generally contains 0.5 to 10% by weight, preferably 3.0 to 8.0% by weight, particulary favourably 4.0 to 6.0% by weight of bile acid. The natural bile extracts may also contain bilirubin, pigments, cholestrol, mucin and minerals.

The pharmaceutical composition according to the invention may contain as bioflavonoid components the following: natural, semi-synthetic or synthetic bioflavonoids of plant or animal origin or the glycosids thereof; extracts prepared of bioflavonoid or its glycosids containing plants or plant parts (such as flower, fruit, leaves, root or any other plant part) in a solvent or dry extracts, or the dry forms of the above plants or plant parts; extracts prepared of bioflavonoid or its glycosid containing animal products in a solvent or in dry form. The plant or animal extracts generally contain 0.001 to 10% by weight, preferably 1 to 8% by weight, particularly favourably 5 to 6% by weight of bioflavonoid (related to the dry substance).

The invention is based on the recognition that the above bile acid components and the bioflavonoid components may have a synergistic interaction to each other, enhance each other's effect and by their simultaneous administration an improved activity can be observed than that of the components administered alone.

The pharmaceutical compositions of the invention can preferably be applied for

improving the functions of the gastrointestinal system and liver-bile duct, consequently for the treatment and/or prevention of gastro-intestinal diseases and diseases of the liver and bile duct (hepatobiliary tract),

the treatment of several diseases associated with gastrointestinal diseases and those of the liver-bile duct,

the improvement of digestion and absorbing of fat-soluble food components, food additives and pharmaceutics and for the treatment of different disorders and diseases associated with the damaged digestion and absorbing of the above fat-soluble substances.

The bile acids are the end products of the cholesterol used synthetized in the liver. The chemical structure of bile acids are very similar to those of the cholesterol and steroid hormones. The carboxyl group of bile acids is conjugated to glycine with amide bond (glycocholic acid) or to taurine (taurocholic acid), and generally forms salt (for example sodium salt) [Northfield, .T., Jazrawi, R, Zentler-Munro, P.: Bile Acids in Heath and Disease: Update on Cholesterol, Gallstones and Bile Acid Diarrhea, Kluwer Academic Publisher, Amsterdam (1988)].

After per oral administration the bile acids enter into the enterobiliary cycle promoting lipid digestion by their detergent effect and reabsorbed from the intestine the bile acids then reach the liver by portal vein and stimulate further bile secretion. Furthermore bile acids are detergents of bacterial enterotoxins (lipopolysaccharides). These lipopolysaccharides are responsible for the clinical symptoms of enteral infections, septic shock and intestinal syndrome of acute radiation disease [Bertok L. Effect of bile acids on endotoxin in vitro and in vivo (physico-chemical defense). Bile deficiency and endotoxin translocation. Ann N Y Acad Sci 30; 851: 408-410 (1998)]. The liposaccharides furthermore activate the inducable nitrogen oxydase (NOS) and the forming of nitrogen oxide (NO) is responsible for the cell damage occurring in case of several systemic diseases (such as mental disorders, neurodegenerative diseases, cardiovascular diseases, retinal degeneration caused by age and degenerative diseases of the osseous-muscular system.

The bioflavonoids are very frequent constituents of plants giving the colors of leaves, flowers and fruits. The chemical constitution of bioflavonoids is characterized by a common C6-C3-C6 skeleton (diphenylpropane or phenyl-benzopyrane). Depending on the highly variable side chains the bioflavonoids can be assigned to the following groups: chalcons, flavonoids, anthocyanidins, isoflavonoids, neoflavonoids and flavonoignans [J. B. Harbone (editor), Herbert Baxter: The Handbook of Natural Flavonoids, John Wiley & Sons, New York (1999)].

In case of digestive diseases the bioflavonoids have antiinflammatory effect. The main pharmacological effect of bioflavonoids originates from the antioxidant property as well as from their inhibitory effect on histamine released from the polymorphonuclear cells and mast cells [DiCarlo G., Autore G, Izzo A A, Maiolino P, Mascolo N, Viola P, Diumo M V, Capasso F-inhibition of intestinal motility and secretion by flavonoids in mice and rats: structure-activity relationship. J. Pharm. Pharmacol, 45: 1054-9 (1993); Medina, S. f., Galvez, J., Romero, J. A, Zarzuelo, A: Effect of quercitrin on acute and chronic experimental colitis in the rat. J. Pharmacol Exp. Ther. 278: 771-9 (1996)]. According to recently published studies the antiinflammatory effect of certain bioflavonoids may come from its inhibitory effect on the generation of proinflammatory cytokines—first of all inducable nitrogen oxydase, phosphodiesterase and protein kinases [Middleton E. Jr., Kandaswami C., Theoharides T C.: The effects of plant flavonoids on mammalian cells: implications for inflammation, heart disease and cancer. Pharmacol Rev 52: 673-751 (2000); Manthey J A., Grohmann K., Guthrie N.: Biological properties of citrus flavonoids pertaning to cancer and inflammation. Curr. Med. Chem. 8: 135-153 (2001)].

The naturally occuring bioflavonoids bind glycosides and other polyphenols which protect the very sensitive bioflavonoids from oxidative and enzymatic damage. For this reason the natural bioflavonoids may be used more preferably than the purified or synthetic bioflavonoids.

The bioflavonoids of natural origin may further contain other pharmacologically compatible substances such as tannic acid, volatile oils, vitamins, hormones, lipids and waxes, mineral salts and trace elements, color substances (chlorophill, xantophill, carotene), amino acids, terpenes (e.g. diterpenes, triterpens, sesquiterpenes), naphtoldianthrone-hypericin, polyphenols (saponone), bactericide substances (allicin).

The pharmaceutical compositions according to the invention may contain as bioflavonoid component preferably the extracts of the following plants:

Betula pendula (silver birch)

Centaurea cyanus (cornflower)

Cynara scolymus (leaves artichoke)

Euphralia roskovina (euphrasy)

Ginkgo biloba (leaves)

Hypericum perforatum (St. John's worth)

Matricaria chamomillae (flower of chamomille)

Melissa officinalis (leaves of balm)

Mentha piperitae (leaves of mint)

Passiflora incarnata (granadilla)

Ribes nigrum (leaves and fruits of ribes or black currant)

Rosmarinus officinalis (leaves and flowers of rosemary)

Salvia officinalis (leaves of saga)

Sylibum marianum (leaves and fruit of lady's thistle)

Vaccinium myrthillus (fruit of huckleberry)

Viola tricolor (viola).

The pharmaceutical compositions of the invention contain bile acid or the salt thereof in a dose of generally 1.0-300 mg/dose, that is 1.0-1000 mg/day; preferably 1.0-100 mg/dose, i.e. 1.0-500 mg/day; especially preferably 1.0-50 mg/dose, i.e. 1.0-200 mg/day.

The pharmaceutical compositions of the invention contain bioflavonoid in a dose of generally 1.0-200 mg/dose, i.e. 1.0-1000 mg/day; preferably 10-200 mg/dose, i.e. 10-400 mg/day; especially favourably 50-200 mg/dose, i.e. 50-400 mg/day.

In the pharmaceutical compositions of the invention the weight ratio of the bile acid component and the bioflavonoid component is a value between 1:10 and 10:1, preferably between 1:3 and 3:1, especially preferably 1:1.

The pharmaceutical compositions of the invention furthermore may contain fat-soluble vitamins, volatile oils, multiple unsaturated fatty acids, fat-soluble hormones, pharmaceutically active substances and food additives. As further component preferably fat-soluble vitamins, particularly favourably vitamin A may be used.

For practical purposes in the present invention the amount of the bile acid component is related to the ursodeoxycholic acid (arbitrarily choosen mass unit is 1.0). The mass/weight ratio of some bile acid components related to ursodeoxycholic acid is as follows: cholic acid 1.2; dehydrocholic acid 1.1; deoxycholic acid 1.0; kenodeoxycholic acid 1.0.

In the present invention the amount of the bioflavonoid component is practically related to quercetine (arbitrarily choosen mass unit is 1.0). The mass ratio of some bioflavonoid components related to quercetine is as follows: chalchone 0.94; flavones 0.94; flavonones 0.94; anthocyanines 0.94; isoflavonoids 0.94; neoflavonoids 0.76.

The pharmaceutical compositions of the invention are prepared by usual methods of the pharmaceutical industry. Bile acid component and the bioflavonoid component are mixed with inert pharmaceutically acceptable carriers and/or auxiliary agents, then the mixture is prepared in galenic form. The pharmaceutical compositions of the invention may be prepared preferably in form of enteric coated tablets or capsules.

When preparing enteric coated dragées at first a core containing bile acid and bioflavonoid as well as pharmaceutical carriers and/or auxiliary substances is prepared. As carrier and/or auxialiary agent the following may be used: calcium carbonate, magnesium carbonate, stearic acid, talc, cellulose derivatives (such as sodium-carboxymethylcellulose), cellulose, starch-like substances (such as potato starch), saccharose, lactose, talc. The core is then coated by an enteric coated coating which does not dissolve in the stomach only in the intestine. For this purpose different acrylic acid esters and methacrylic acid esters (such as Eudragit) or cellulose-acetyl-phthalate (CAP) are used

The compositions of the invention may be prepared furthermore in form of hard or soft gelatine capsules by using the usual methods of the pharmaceutical industry.

The pharmaceutical compositions of the invention may be used furthermore in usual manner in form of intravenous or intramuscular injection or eye drops.

The synergistic effect of the pharmaceutical compositions according to the invention are proved by the following experiments.

1. Treatment or Prevention of Digestive Disorders

In an open clinical study it was found that by administering bile acid and bioflavonoid together (composition according to Example 2) the symptoms of digestive disorders (satiety, meteorism, pain, nausea) improve. The patients treated with the composition of the invention during or at the end of the meals were able to digest any type of foods including heavy or spicy ones. The psychosomatic condition of the patients significantly improved. The improvement of the symptoms of patients treated with the composition of Example 2 was higher than that of the bile acid or bioflavonoid treatment alone.

In these studies 48 patients affected by either non-ulcer dyspepsia, biliary dyskinesis or irritable bowel syndrome were involved. 24 patients with unrestricted fat diet received only 150 mg of ursodeoxycholic acid (group A) and 24 patients only bioflavonoid (300 mg of quercetin) (group B) for 3 months. After one-month washout both groups received ursodeoxycholic acid+quercetin (Example 2) (group C) for another 3 months.

The overall (psychosomatic) condition and subjective symptoms were compared to the results of an age and sex matched control group.

The evaluation of the psychosomatic condition: improved, unchanged, worsened.

The gastrointestinal symptoms (postprandial early satiety, abdominal distention or bloating, nausea, vomitus, diarrhea, constipation) were evaluated by using the following grading system:

0=no symptoms

1=mild

2=moderate

3=severe.

The results obtained are summarized in the following Table 1.

TABLE 1

| Symptoms | Group A | Group B | Group C |
|---|---|---|---|
| Improved | 67% | 45% | 89% |
| Unchanged or worsened | 33% | 55% | 11% |

In the bile acid treated group A the overall condition improved in 67% of the cases, while remained unchanged or worsened in 33%.

In the bioflavonoid treated group B the overall condition improved in 45% of the cases and remained unchanged or worsened in 55%.

In the group C treated with the composition of Example 2 the physchosomatic condition improved in 89% o the cases, while remained unchanged or worsened in 11%.

The data of the above Table prove that—compared to administering each component alone—the composition of the invention significantly improves the symptoms, consequently the composition has synergistic effect.

2. Evaluation of Subjective Symptoms

The treatments A, B and C shown in point 1 were used. The results are shown in Table 2. In Table 2 the average complaint ratio related to one patient was given (average complaint ratio=total number of points/number of patients). The values of the control groups are given in brackets.

TABLE 2

| Symptoms | Group A | Group B | Group C |
|---|---|---|---|
| Satiety | 2.06 (2.92) | 2.04 (2.86) | 0.46 (2.74) |
| Meteorism | 2.12 (2.78) | 1.96 (2.33) | 0.51 (2.67) |
| Nausea | 0.83 (1.17) | 0.92 (1.25) | 0.42 (1.08) |
| Vomitus | 0.12 (0.83) | 0.46 (0.75) | 0.13 (0.71) |
| Diarrhoea/ | 2.19 (2.54) | 0.63 (2.54) | 0.29 (2.67) |
| constipation | | | |

In case of the group treated with the composition of Example 2 all symptoms moderated significantly better than by administering the components alone. The synergism is thus proved

3. Improvement of Activity of Fat-Soluble Substances

72 female patients affected by digestive disorders, “dry eye” syndrome and premenstrual syndrome were treated Intramuscularly administered vitamin A improved the symptoms of “dry eye” and premenstral syndrome, but it was ineffective by peroral administration. It was found that administering the composition of the invention orally together with vitamin A the symptoms improve nearly in a manner than by administering vitamin A intramuscularly.

The subjective symptoms were evaluated by questionairs by using the following grade:

0=no symptoms

1=mild

2=moderate

3=severe.

“Dry eye” syndrome has the following symptoms: sensation of dryness, foreign body, tired eye, soreness, scatchiness, burning, reduced tearing of emotion, difficulties of opening the eye upon waking, episodes of excessive tearing, particular sensibility to wind, air conditioning, smoking, topical medication. Objective signs: particulate matter on the ocular surface, mucusthread at the inner canthus or in the lower fornix, deposits at the orefices of the meibomian glands, filamentary keratitis, hyperemia of the bulbar conjunctiva, papillary hypertrophy of the tarsal conjunctiva, thin tear meniscus, decreased brightness of the bulbar conjunctiva, frequent blinking, tear film abnormalities with fluorescein staining.

The patients were treated as follows:

Treatment A: 300.000 IU vitamin A in oil intramuscular administration ones per months,

Treatment B: 50.000 IU vitamin A acetate dragée peroral administration, once every second day,

Treatment C: 50.000 IU vitamin A acetate dragée peroral administration, once every second day+composition of the invention (Example 4).

The results are summarized in Table 3.

TABLE 3

| Symptoms | Treatment A | Treatment B | Treatment C |
|---|---|---|---|
| Improved | 80% | 12% | 76% |
| Unchanged | 20% | 88% | 24% |

The above data show that the “dry eye” symptoms improved only weakly by administering vitamin A perorally, while by peroral administration of vitamin A and the composition of the invention together the dry eye symptoms improved significantly. This improvement almost reach those shown by intramuscular administration of vitamin A.

4. Treatment of Premenstrual Symptoms

Symptoms of the premenstrual syndrome: food (sweets or chocolate) craving, exhaustion, irritability, fearfullness, anxiety or depression, hostility, feeling out of control, confusion, sleep disturbances, acne, fluid retention, weight gain, breast swelling, aches and pains, bloating, constipation or diarrhoea.

The number of patients, methods of treatment and the grade of evaluation were the same as shown in experiment 3.

The results are summarized in Table 4.

TABLE 4

| Symptoms | Treatment A | Treatment B | Treatment C |
|---|---|---|---|
| Improved | 72% | 8% | 84% |
| Unchanged | 28% | 92% | 16% |

Table 4 proves that the composition of the invention significantly improves the effect of the treatment with vitamin A. The symptoms hardly improve by peroral administration of vitamin A, while by peroral administration of the composition according to the invention together with vitamin A a significant improvement of the symptoms occurs, which exceeds the effect of vitamin A administered intramuscularly. Thus the synergism is proved.

5. Topical Treatment of “Dry Eye”

Dry eye model was created in 16 Wistar rats in one eye by retrobulbar injection of capsaicin as it was published in [Camras PCB, Bito L Z: The pathophysiological effects of nitrogen mustard on the rabbit eye. II. The inhibition of the initial hypersensitive phase by capsaicin and the apparent role of substance P. Invest Ophthaimol Vis Sci. 19, 423-8 (1980)], while the other eye (control) was not treated at all.

The following treatments were applied:

A treatment: 4 rats were treated with topical artificial tear drops, one drop 3 times per day;

B treatment: 4 rats were treated with the composition of Example 10, one drop 3 times per day,

C treatment: 4 rats were treated with the composition of Example 10, one drope 3 times per day+one drop of vitamin A 3 times per day (oil drop; 30 000 IU/ml),

D treatment: 4 rats were treated with vitamin A, one drop 3 times per day (oil drop 30 000 IU/ml).

The condition of eyes were registered as written in the literature and evaluated 4 weeks after treatment on the following scale:

0=no alteration;

1=punctiform ephitelial loss of the cornea;

2=large ephitelial loss of the cornea with mild corneal edema;

3=large ephitelial loss of the cornea with severe corneal edema.

The results were summarized in the following Table 5.

TABLE 5

| Symptoms | Treatment B | Treatment C | Treatment D |
|---|---|---|---|
| Reduced eye alteration | 50% | 67% | <5% |
| compared to treatment | | | |
| with artificial tear drop | | | |
| (treatment A) | | | |

The topical application of the composition of the invention (treatment B) the improvement of eye alteration compared to group A is 50%. In case of topical treatment D only with vitamin A the improvement is not significant. In case of group C, when treatment is carried out by combining the composition of the invention and vitamin A the improvement is 67%, i.e. exceeds the values obtained by the group treated with vitamin A alone.

6. Topical Treatment of Premenstrual (Acne) Skin

In three consecutive menstrual cycles, started on the 5th day before the expected day of menstruation an ointment of the following composition was applied in a thin layer on the skin of face of 8 voluntary female patients, once in the evening for 10 days:

treatment A: 30 000 IU vitamin A (=1 ml oil)+25 g Ung. simplex

treatment B: composition according to Example 11.

The test results are summarized in Table 6.

TABLE 6

| | Treatment A | Treatment B |
|---|---|---|
| Complaints of the patients | 100% | 40% |
| compared to treatment A | | |

As a result of the composition of the invention the complaints of the patients reduced by 60% compared to those treated with a composition containing vitamin A only.

The bile acid components described in the Examples can be purchased in trade.

Further details of the invention are described in the following non-limiting Examples.

### EXAMPLE 1

Enteric coated dragées having the following composition are prepared by using the usual methods of pharmaceutical industry:

| Component | Amount mg/dragée |
|---|---|
| Ursodeoxycholic acid | 150.00 |
| Quercetin | 150.00 |
| Cedra carnauba | 0.02 |
| Cera alba | 0.01 |
| Sodium-carboxy-methyl-cellulose | 0.40 |
| Polyvidon | 8.50 |
| Stearic acid | 5.00 |
| Calcium carbonate | 15.00 |
| Eudragit L | 11.00 |
| Eudragit S | 4.20 |
| Cellulose | 21.00 |
| Colloidal siliciumdioxide | 8.80 |
| Potato's starch | 9.50 |
| Saccharose | 60.00 |
| Lactose | 81.00 |
| Talc | 22.50 |
| Ariavit-green | 0.20 |
| Total weight: | 550.00 |

### EXAMPLE 2

Hard gelatine capsules of the following composition are prepared by using the usual methods of pharmaceutical industry:

| Components | Amount, mg/capsule |
|---|---|
| Ursodeoxycholic acid | 150.00 |
| Quercetin | 300.00 |
| Lactose | 900.00 |
| Starch | 150.00 |
| Magnesium-stearate | 15.00 |
| Total weight: | 1515.00 |

### EXAMPLE 3

Enteric coated dragées of the following composition are prepared by the usal methods of pharmaceutical industry:

| | |
|---|---|
| Components | Amount, mg/dragée |

| | | |
|---|---|---|
| Animal bile extract (dry weight) | 50.00 | |
| Passiflora in carnata extract | 200.00 | |
| (dry weight) | | |
| Allium sativum extract (dry weight) | 100.00 | |
| Vitamin A | 3000 | IU |
| Carbo medicinalis | 50.00 | |

The carriers and auxiliary agents are the same as given in Example 1.

### EXAMPLE 4

Hard gelatine capsules of the following composition are prepared by using the usual methods of the pharmaceutical industry:

| Components | Amount, mg/capsule |
|---|---|
| Ursodeoxycholic acid | 150.00 |
| Passiflora incarnata extract (dry weight) | 300.00 |
| Melissa officinalis (dry weight) | 100.00 |

The carriers and auxiliary agents are the same as given in Example 2.

### EXAMPLE 5

Enteric coated capsules having the following composition are prepared by usual methods of the pharmaceutical industry:

| | |
|---|---|
| Components | Amount, mg/capsule |

| | | |
|---|---|---|
| Animal bile extract (dry weight) | 100.00 | |
| Passiflora incarnata extract | 200.00 | |
| (dry weight) | | |
| Vaccinium Mirthyllus (dry weight) | 200.00 | |
| Ginkgo biloba extract | 200.00 | |
| Vitamin A | 3000 | IU |

The carriers and auxiliary substances are given in Example 2.

### EXAMPLE 6

Enteric coated capsules of the following composition are prepared by usual methods of the pharmaceutical industry:

| | |
|---|---|
| Components | Amount, mg/capsule |

| | | |
|---|---|---|
| Animal bile extract (dry weight) | 100.00 | |
| Passiflora incarnata extract (dry weight) | 200.00 | |
| Hypericum perforatum extract | 100.00 | |
| Vitamin A | 3000 | IU |

The carriers and auxiliary agents are the same as given in Example 2.

### EXAMPLE 7

Enteric coated capsules of the following composition are prepared by usual methods of the pharmaceutical industry:

| | |
|---|---|
| Component | Amount, mg/capsule |

| | | |
|---|---|---|
| Animal bile extract (dry weight) | 100.00 | |
| Passiflora incarnatum extract (dry weight) | 200.00 | |
| Salvia officinalis extract (dry weight) | 100.00 | |
| Melissa officinalis extract (dry weight) | 100.00 | |
| Vitamin A | 3000 | IU |

The carriers and auxiliary agents are the same as described in Example 2.

### EXAMPLE 8

Soft gelatine capsules of the following composition are prepared by usual methods of the pharmaceutical industry:

| | |
|---|---|
| Components | Amount, mg/capsule |

| | | |
|---|---|---|
| Glycodeoxycholic acid | 20.00 | |
| Passiflora incarnatum (dry weight) | 100.00 | |
| Fish oil | 750.00 | |
| Vitamin A | 3000 | IU |
| Vitamin E | 10.00 | |

### EXAMPLE 9

An aqueous solution of the following composition are prepared by usual methods of the pharmaceutical industry:

| Components | Amount, mg/10 ml solution | |
|---|---|---|
| Tauro-ursodeoxycholic acid | 10.00 | |
| Euphrasia rostkovina extract | 100.00 | |
| (dry weight) | | |
| Centaurea cyanus extract (dry weight) | 100.00 | |
| Vitamin A | 30 000 | IU |
| Physiological salt solution | 10 | ml |

### EXAMPLE 10

Eye drop of the following composition is prepared by usual methods of the pharmaceutical industry:

| Components | Amount, mg/1 ml solution | |
|---|---|---|
| Tauro-ursodeoxycholic acid | 1.00 | |
| Euphrasia rostkovina extract | 10.00 | |
| (dry weight) | | |
| Centaurea cyanus extract (dry weight) | 10.00 | |
| Rutin | 10.00 | |
| Physiological salt solution | 1 | ml |

### EXAMPLE 11

An ointment of the following composition is prepared by usual methods of the pharmaceutical industry:

| Components | Amount, mg/10 g of ointment | |
|---|---|---|
| Tauro-ursodeoxycholic acid | 50.00 | |
| Viola tricolor extract (dry weight) | 100.00 | |
| Betula pendula extract (dry weight) | 100.00 | |
| Fumaria officinalis (dry weight) | 100.00 | |
| Vitamin A | 30 000 | IU |
| Unguentum simplex | 10 | g |

1. A pharmaceutical composition containing as active ingredient a bile acid component and a bioflavonoid component together with pharmaceutically suitable carriers and/or auxiliary agents.

2. A pharmaceutical composition according to claim 1 containing bile acid as bile acid component.

3. A pharmaceutical composition according to claim 1 containing bile acid conjugate or semi-synthetic or synthetic analogue or a bile acidic salt as bile acid component.

4. A pharmaceutical composition according to claim 1 containing as bile acid component a semi-synthetic or synthetic bile acid-containing bile component.

5. A pharmaceutical composition according to claim 4 containing as bile acid component complete animal bile, the extract of animal bile dry or in solvent or the bile acid containing fraction of animal bile.

6. A pharmaceutical composition according to claim 2 containing the sodium salt or the conjugate of bile acid formed with taurine or glycine.

7. A pharmaceutical composition according to claim 1 containing as bile acid component ursodeoxycholic acid, sodium-deoxycholate, tauro-deoxycholate, glycodeoxycholate, sodium-ursodeoxycholate, glyco-ursodeoxycholate or tauro-ursodeoxycholate.

8. A pharmaceutical composition according to claim 1 which contains as bioflavonoid component a bioflavonoid-containing natural substance of plant or animal origin or the glycosides or extracts thereof dry or in solvent, or semi-synthetic or synthetic bioflavonoid or the glycosides thereof.

9. A pharmaceutical composition according to claim 1 which contains bile acid and a bioflavonoid component according to claim 8.

10. A pharmaceutical composition according to claim 9 also containing a fat-soluble substance.

11. A pharmaceutical composition according to claim 10 containing vitamin A.

12. A pharmaceutical composition according to claim 1 to 11 containing a bile acid component and a bioflavonoid component in a weight ratio 1:10-10:1.

13. A pharmaceutical composition according to claim 12 containing the bile acid component and the bioflavonoid component in a weight ratio 1:1.

14. A pharmaceutical composition according to any of claims 1 to 13 in a form of enteric coated tablets or capsules or intravenous or intramuscular injection or eye drop.

15. A process for preparing a pharmaceutical composition of claim 1 characterized in that a bile acid component and a bioflavonoid component are mixed with pharmaceutically suitable carriers and/or auxiliary substances and put in a form suitable for medical use.

16. Use of the mixture of a bile acid component and a bioflavonoid component for preparing pharmaceutical compositions.

17. Use of the mixture of a bile acid component and a bioflavonoid component for preparing pharmaceutical compositions enhancing the function of the digestive system, and suitable for the treatment or prevention of diseases associated with the damaged digestion or absorption of fat-soluble food, as well as for improving the eye and premenstrual symptoms.

18. A pharmaceutical composition according to claim 1 containing a bile acid component and a bioflavonoid component for improving the topical activity of fat-soluble substances.
